(19) United States
(12) Patent Application Publication
(10) Pub. No.: US 2017/0203116 A1
(43) Pub. Date: Jul. 20, 2017

(54) POWER MANAGEMENT IN TRANSCRANIAL MAGNETIC STIMULATORS

(71) Applicant: John W. SADLER, Belmont, CA (US)

(72) Inventor: John W. SADLER, Belmont, CA (US)

(21) Appl. No.: 15/249,278

(22) Filed: Aug. 26, 2016

Related U.S. Application Data
(63) Continuation of application No. 13/512,496, filed on Sep. 17, 2012, now abandoned.
(60) Provisional application No. 61/303,188, filed on Feb. 10, 2010.

(51) Int. Cl. A61N 2/00; A61N 2/02

(52) U.S. Cl. CPC A61N 2/006; A61N 2/02

(57) ABSTRACT

A system for supplying high voltage direct current for Transcranial Magnetic Stimulation (TMS) devices under program control features a rate input that commands the power supply to charge the discharge capacitors at a fixed rate until a programmed target voltage is achieved. This reduced demand spikes to the AC line when operating the TMS device, and may allow the TMS device to operate from a lower-rated AC circuit than would otherwise be possible.

### INCORPORATION BY REFERENCE

All publications, including patents and patent applications, mentioned in this specification are herein incorporated by reference in their entirety to the same extent as if each individual publication was specifically and individually indicated to be incorporated by reference.

### FIELD OF THE INVENTION

The present invention relates to systems and methods for providing electrical power to transcranial magnetic stimulation (TMS) systems.

### BACKGROUND OF THE INVENTION

Multi-coil repetitive Transcranial Magnetic Stimulation (rTMS) presents significant power demands that are not easily met by conventional building wiring. A single Magstim Rapid®, for example, may require two 20 Amp circuits to power a single coil. Because each stimulator draws a large surge of power after each discharge, instantaneous power demand on the AC line spikes well above the average demand, resulting in the need to dedicate one or more AC circuits to each stimulator.

A conventional magnetic stimulator consists of a stimulating coil, a discharge capacitor connected to the coil via an electronic switch, and a high voltage DC power supply connected to the capacitor, possibly with series inductors between the power supply and the capacitor, and between the capacitor and the electronic switch. There may also be snubbers or other components designed to protect the capacitor and switches from transient voltages that occur when coil current is switched off. There may also be provision for recovering some of the energy from the coil after discharge and pumping it back into a storage capacitor. There may be additional capacitors for the purpose of storing energy to allow fast recharging of the discharge capacitor.

In use, the DC power supply charges the discharge capacitor rapidly to a predetermined voltage. When triggered, the electronic switch dumps the capacitor's charge into the coil and any series inductors, generating a powerful transient magnetic field. The series combination of coils and capacitors may form a resonant circuit makes it possible for the field to oscillate for several cycles if the switch is closed long enough to allow it. Many stimulators provide options for several different pulse shapes and amplitudes, specifically: biphasic, in which the field does one complete oscillation cycle and then stops, monophasic, in which the field does a half cycle, and polyphasic, in which the field does several oscillations. More exotic waveforms have been described as well, including “theta bursts” comprising pulses of 50 Hz oscillations spaced at 100mS intervals. The period of each magnetic pulse is largely determined by the total inductance of the stimulating coil and any inductors in series with it, and by the capacitance of the discharge capacitor, with resonant frequency Fₜ given (assuming negligible resistance in the circuit) by:

$${F}_{t} = \frac{1}{2 π \sqrt{LC}}$$

In a typical TMS unit, the coil has an inductance (L) of about 15-25 uH, and the capacitor has capacitance (C) of 5-50 uF, resulting in a resonant frequency of 10-20 KHz. It is well-known in the art to use other values to give other resonances as needed.

In most cases, the circuit involves one or more reservoir capacitors and switches that controllably dump charge into the resonant circuit . Once charge is dumped and resonance ceases (or the switch is opened), the capacitor presents a large load to the high voltage supply. The high voltage supply in turn presents a large instantaneous load to the AC line, and because of the nonlinear behavior of the rectifiers in line with the capacitor, the power factor can be 0.6 or less. TMS devices typically fire at rates of from 1 Hz for down-regulation, to 10 Hz or more for up-regulation, and each coil typically dissipates about 40 W of average power at a 1 Hz pulse rate. Because of the pulsed nature of the discharge, it is possible for the stimulator to present an instantaneous load that demands the full capacity of the AC circuit for hundreds of milliseconds as the capacitor begins to charge.

In U.S. Application 2008/0306326, Epstein describes combining an AC power supply and battery to allow a stimulator to generate pulses that otherwise need more power than a standard AC circuit can provide. This solution is limited in that it does little to regulate the power factor or the peak load demanded by the stimulator. Batteries have relatively high effective series resistance as compared to capacitors, and thus are not ideally suited for providing rapid bursts of very high current required by a TMS coil.

In U.S. Application 2007/0293916, Peterchev describes [0054] a programmable charger that allows an operator to set two independent target voltages for a pair of discharge capacitors. The invention is primarily concerned with inducing rectangular electric field pulses into a body organ, and does not address AC line power management.

It would be therefore be desirable to have a magnetic stimulator that presents less supply ripple so that its instantaneous power demand is much closer to its average power demand. This would make it practical to run several stimulators and coils off the same AC line.

### BRIEF DESCRIPTION OF THE DRAWINGS

FIG. 1 is a block diagram of a high voltage power supply, controller, and stimulator coil illustrating aspects of the invention.

FIG. 2 is an exemplary schematic of a high voltage supply showing aspects of the invention.

FIG. 3 illustrates an aspect of the invention in which several coils and corresponding discharge capacitors share one high voltage supply.

FIG. 4 illustrates an aspect of the invention in which several discharge capacitors share one high voltage supply and one coil.

### DETAILED DESCRIPTION OF THE INVENTION

The systems and methods described herein are directed to power supplies and power management for transcranial magnetic stimulation systems.

In one aspect, we provide a TMS system with a programmable high voltage power supply that charges the capacitor at a constant current until it reaches a predefined voltage or total stored energy:

$$E = \frac{{CV}^{2}}{2} or E = \frac{QV}{2}$$

where V is the voltage across the capacitor of capacitance C, and Q is the stored charge in the capacitor at voltage V.

Thus to compute the stored energy one must measure voltage V and either capacitance C of the capacitor or current during the charge cycle.

In another aspect, a multi-coil stimulator consists of one high-voltage supply with several discharge capacitors multiplexed to it via electronic switches and or diodes. There may be passive filtering elements to help manage load spikes between the capacitors and the supply, and snubbers to protect the switches from voltage spikes that arise from rapid switching of inductive loads.

The supply may be configured to charge all capacitors at once using a constant current charging profile until they reach a predefined threshold voltage. The programmed current and threshold voltage may be selected in accordance with a desired firing rate of the stimulator. For example, if the stimulator is to fire at 1 Hz, the supply may be programmed so that each of the n discharge capacitors is charged to the threshold voltage in 1/n second. For a capacitance C and voltage threshold V, the amount of charge Q (in Coulombs) required to charge the capacitor to the threshold voltage is given by:

Q=CV

Thus the average current required to charge the capacitor to threshold in 1 second is Q Amperes, (since an Ampere is defined as one Coulomb per second) and the average current required to charge it in 1/n second is nQ Amperes.

The input switches may be configured to switch the High Voltage (hereinafter abbreviated as HV) supply output rapidly from one capacitor to the next until all capacitors are charged just prior to the firing event.

It would be desirable to interpose a passive filter between the HV supply and the load switches.

The power supply may be a switching type with programmable output voltage, or it may use a multitap transformer in which the output voltage is programmably stepped from a relatively low voltage at the beginning of the charge cycle in steps to the threshold voltage at the end of the charge cycle. Alternatively a single HV supply may charge an array of capacitors through a distributor and a filter inductor. By switching among the capacitors at a rapid rate, the supply ripple is reduced. By providing many more capacitors than the number of coils, and switching banks of capacitors in parallel (possibly with diode logic), it is possible to further reduce supply ripple.

The power supply may have, an input that controls the average charge rate of the load, and another input that controls the threshold voltage.

There may be one HV supply per coil, each designed to draw an average load based on a programmed charging rate as noted above, or a single supply may be multiplexed via switches, diodes, or passive filters to several discharge capacitors.

Multiple discharge capacitors may be connected via switches to a single coil to allow bursts of rapid pulses, and still provide a reduced ripple load profile to the AC line.

Power factor compensation may be added to the HV supply in order to further reduce load harmonics on the AC line. Power factor is the ratio of apparent power (the voltage-current product) to the work done by the load, and is a measure of the degree to which the load appears to the supply as a pure resistive load. AC to DC converters, particularly switching ones, have nonlinear current characteristics that present low power factor loads to the AC line. Power factor may be compensated by analog filtering with high current inductors, or in a switching supply by adding a boost converter in series that is designed to maintain a constant output voltage while drawing a load that is matched to the input waveform as closely as possible.

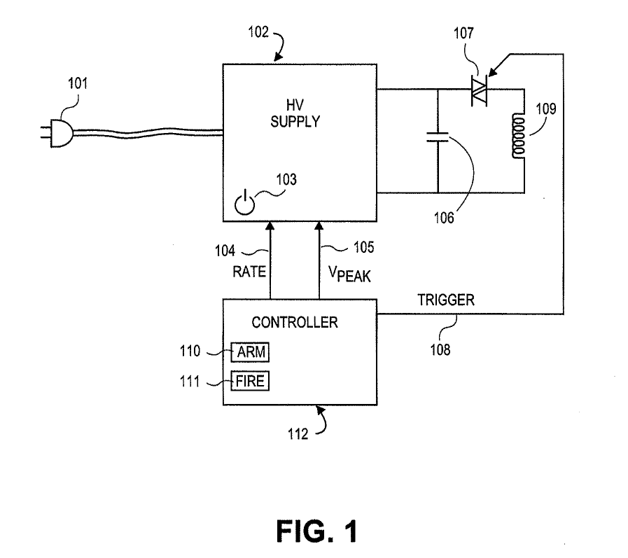

Referring now to FIG. 1, in one aspect of the invention an exemplary single coil TMS system comprises AC line connection 101, High Voltage supply 102 with power switch 103, discharge capacitor 106, stimulating coil 109, and electronic switch 107. Electronic switch 107 is shown as a TRIAC, but may be any switching device with sufficiently high current and blocking voltage and short switching time. Suitable devices include TRIACS, thyristors (also known as SCRs), Insulated Gate Bipolar Transistors, and power MOSFETs, among others. Electronic switch 107 may have a snubbing network consisting of a resistor and a capacitor connected across its load terminals to ensure correct turn-off because of the inductive load.

Still referring to FIG. 1, High Voltage Supply 102 has two inputs: Rate input 104 controls the rate of charge of capacitor 106, and Peak Voltage input 105 controls the maximum charge voltage to be delivered to capacitor 106. Controller 112 may determine these values by user input or program control, or a combination of the two. For example, the charging rate signal delivered to Rate input 104 may be a function of the rate at which the stimulating coil is to be pulsed, and Peak Voltage signal delivered to 105 may be a function of the peak field strength desired. This is sometimes set with reference to the field strength required to stimulate the motor cortex of the subject under treatment. Note that Rate signal 104 and Peak Voltage signal 105 may be communicated over a digital bus or via analog signaling means. The signals are shown as two separate lines for clarity of illustration only. As shown, controller 112 also comprises Trigger output 108, which controls firing of electronic switch 107. Alternatively, controller 112 may provide a trigger signal to HV supply 102, which may in turn control the gate input of electronic switch 107. This alternative architecture may allow HV supply 102 to adapt its charge rate to the actual pulse rate, or to delay firing until capacitor 106 has reached the peal voltage programmed by Peak Voltage signal 106.

Still referring to FIG. 1, Controller 112 optionally comprises a manual Arm switch 110 and a manual Firing switch 111. Controller 112 may also have a not shown digital input for control by an external processor or computer. Controller 112 may comprise a processor and a stored program for delivering stimulus pulses for one or more applications. In this case controller 112 may also comprise a display or a set of status indicators as well as a keypad or other means well-known in the art for receiving input from the clinician operating the device. Such input may for example include selection of a particular treatment mode from a menu of possibilities, a way to calibrate motor threshold—the peak voltage at which the subject's motor cortex responds to the stimulus pulse by muscle twitching, and buttons or switches to allow the clinician to start and stop stimulus pulses.

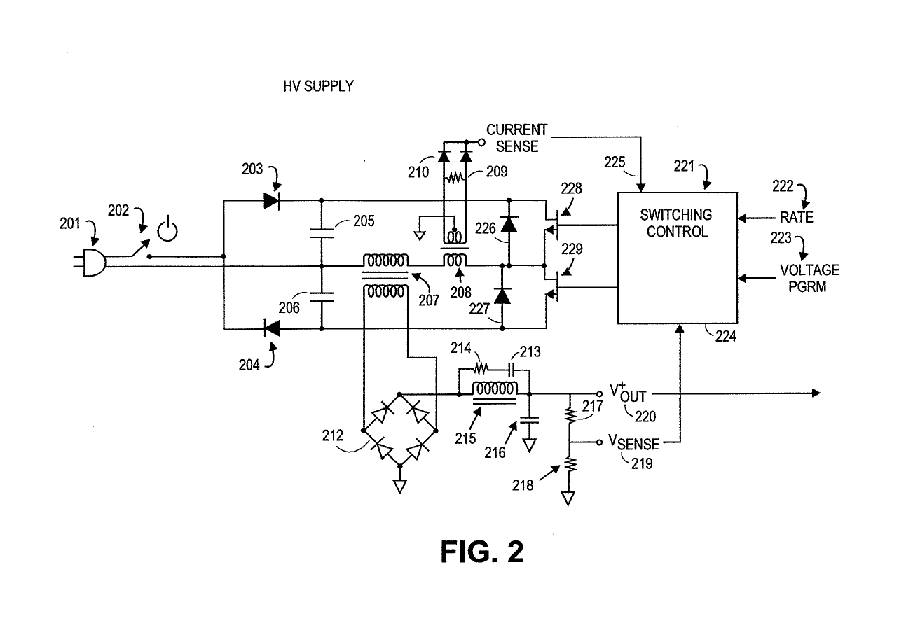

FIG. 2 shows an exemplary schematic of a high voltage supply comprising aspects of the invention, particularly using one HV supply with programmable charge rate to charge several discharge capacitors at a controlled rate so that demand to the AC line is leveled according to a programmed pulse rate. AC line connection 201 feeds power via line switch 202 to a rectifier section comprised of diodes 203 and 204 and filter capacitors 205 and 205, providing positive and negative filtered DC for switching devices 228 and 229. Switching devices 228 and 229 drive power transformer 207. Current transformer 208 senses the current flowing in the primary winding of power transformer 207. Diodes 210 rectify the current flowing from the secondary winding of current transformer 208 generate a current-sense signal 211 that feeds Current Sense input 225 of switching control 221. Resistor 209 limits the peak voltage across the secondary of current transformer 208. Switching control 221 uses current sense input 225 as part of a feedback control to regulate the width of pulses sent to switching devices 228 and 229, thus controlling the rate of charge of the load.

Still referring to FIG. 2, Switching devices 228 and 229 may be power MOSFETs or IGBTs, among other choices well-known in the art. Snubber diodes 226 and 227 prevent reverse voltage from transformers 207 and 208 from damaging switching devices 228 and 229. Switching devices 228 and 229 receive gate control from switching controller 221, which typically generates square-wave pulses with variable duty cycle to develop an AC waveform across power transformer 207 and current transformer 208.

Still referring to FIG. 2, the secondary winding of power transformer 207 delivers high voltage AC to bridge rectifier 212, the output of which is filtered by inductor 215 and capacitor 216 to deliver filtered high voltage direct current to output 220. A snubbing network consisting of capacitor 213 and resistor 214 serves to limit voltage spikes across inductor 215. A potential divider consisting of resistors 217 and 218 delivers voltage sense signal 219 to controller 221. Controller 221 determines the duty cycle of control signals to switching devices 228 and 229 based on peak voltage input 223 and rate input 222.

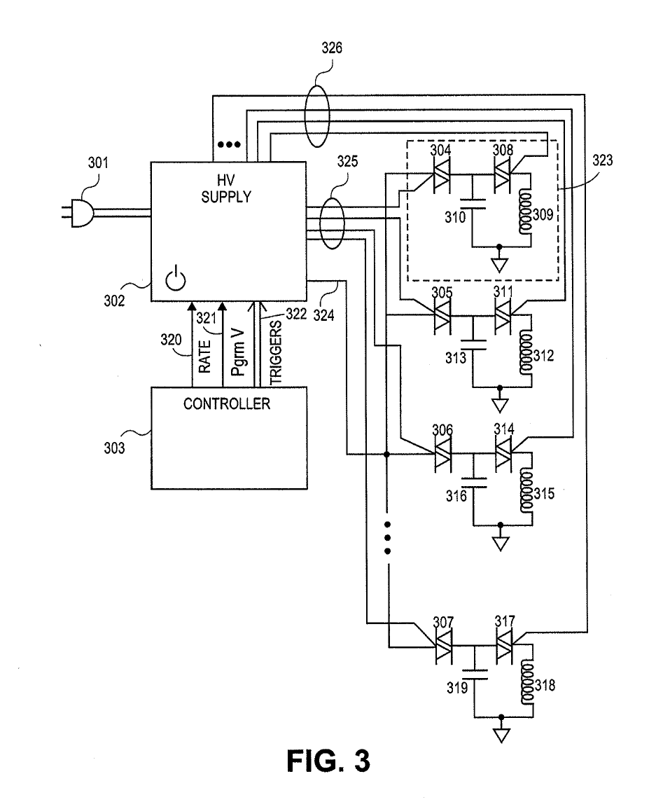

FIG. 3 illustrates another aspect of the invention. In this case High Voltage supply 302 is connected to several coil modules, each comprising a discharge capacitor, coil, trigger, and charging gate switch. The triggers (308, 311, 314, 317) are shown as TRIACS for illustrative purposes with the understanding that other suitable devices as discussed above may be used. The charging gates (304-307) are shown as TRIACs for illustrative purposes, assuming that HV supply 302 delivers power in pulses rather than as continuous DC so that the charging gate TRIACs shut off after each pulse. If power from HV supply 302 is instead delivered as DC, it is necessary to use switching devices that do not latch on, such as power MOSFETs or IGBTs. Power output 324 delivers DC or pulsed DC power to the charging gates.

Still referring to FIG. 3, a first module 323 comprises charging gate 304, capacitor 310, trigger 308, and coil 309. A second module comprises charging gate 305, capacitor 313, trigger 311, and coil 312. A third module comprises charging gate 306, capacitor 316, trigger 314, and coil 315. A fourth module comprises charging gate 307, capacitor 319, trigger 317, and coil 318. High voltage supply 302 has a gate output (325 as a group) and a trigger output (326 as a group) for each module. Each gate output controls the flow of charge current to the corresponding module's discharge capacitor, and each trigger output discharges its corresponding capacitor into the coil.

HV supply 302 may enable each charge gate in turn until its respective capacitor is fully charged, or may switch among all capacitors in rapid bursts, resulting in a roughly uniform rate of charging of all capacitors. Controller 303 is shown with at least three outputs to high voltage supply 302. Rate output 320 determines charge current as described above; Peak Voltage output 321 controls the maximum voltage delivered to the capacitors—this limits the maximum amount of energy delivered to a coil, and is typically set by the clinician with reference to the power level that elicits a motor response when a single coil is positioned over the motor cortex; one or more Trigger signals 321 function as described above to control the release of energy into a stimulus coil. Trigger signals 321 may consist for example of one signal line for each coil, one signal line for each discharge capacitor, an address bus that selects a specific capacitor or coil for discharge and a separate signal to fire it, a single line that fires all coils at once, or any other method well-known in the art for addressing and triggering multiplexed elements under program control.

Still referring to FIG. 3, while four stimulating modules are shown for illustrative purposes, any number could be used according to the invention). Alternatively, each coil may be connected to more than one discharge capacitor via an independent trigger and charging gate. This may allow the system to deliver bursts of pulses, or to allow bidirectional pulses by connecting capacitors with opposite polarities to the coil. Further optional aspects of the device may include energy recovery circuits that partially recharge a discharge capacitor by harvesting residual energy stored in the coil at the end of a pulse.

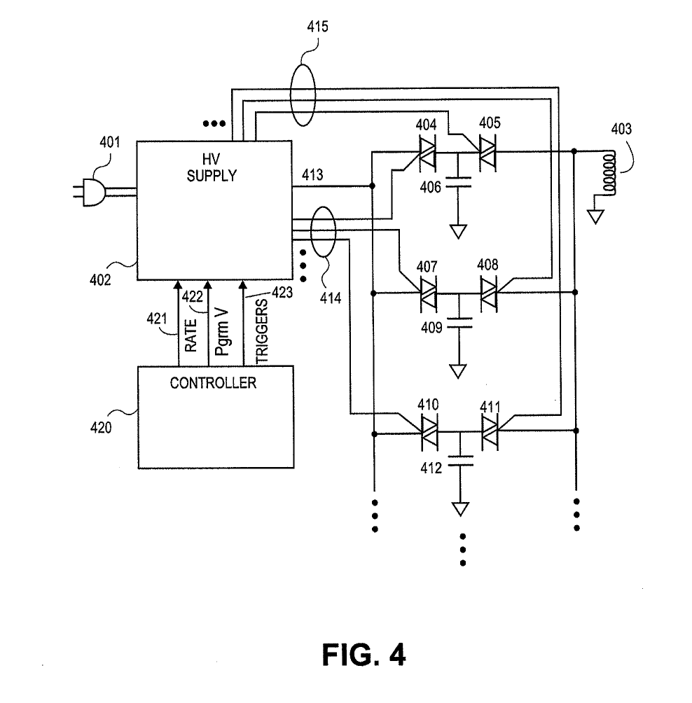

Referring now to FIG. 4, another aspect of the invention allows several discharge capacitors to be mapped to each coil. For clarity FIG. 4 shows one coil 403, with three capacitor modules connected to it. Note that any combination of capacitors and coils may be realized, as long as there is at least one capacitor per coil. A first capacitor module comprises charging gate 404, capacitor 406, and trigger 405. A second capacitor module comprises charging gate 407, capacitor 409, and trigger 408. A third capacitor module comprises charging gate 410, capacitor 412, and trigger 411. In this configuration HV supply 402 charges the capacitors by switching charge gates in sequence as described previously. By multiplexing several discharge capacitors to one coil, a rapid burst of stimulus pulses is possible.

As for additional details pertinent to the present invention, materials and manufacturing techniques may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

What is claimed is:

1. A power supply for a transcranial magnetic stimulator that comprises a current programming input such that the output current of the power supply is determined by the current programming input.
